(19) United States
(12) Patent Application Publication
(10) Pub. No.: US 2020/0196934 A1
(43) Pub. Date: Jun. 25, 2020

(54) POSTURAL SWAY ANALYSIS SYSTEM AND METHOD

(71) Applicant: Purdue Research Foundation, West Lafayette, IN (US)

(72) Inventors: Babak Ziaie, Lafayette, IN (US); Shirley Rietdyk, West Lafayette, IN (US); Albert Kim, Portland, OR (US); Junyoung E Kim, Lafayette, IN (US)

(73) Assignee: Purdue Research Foundation, West Lafayette, IN (US)

(21) Appl. No.: 16/208,524

(22) Filed: Dec. 3, 2018

Related U.S. Application Data
(60) Provisional application No. 62/593,679, filed on Dec. 1, 2017.

(51) Int. Cl. A61B 5/00; A61B 5/11

(52) U.S. Cl. CPC A61B 5/4023; A61B 5/0077; A61B 5/1127; A61B 5/1117; A61B 5/6898; A61B 5/0013; A61B 5/7275; A61B 2560/0223; A61B 2576/00; A61B 5/706

(57) ABSTRACT

A postural sway analysis system is disclosed. The system includes a camera worn by an individual, a processing unit coupled to the camera, a floor marker placed on a floor near the shoes or feet of the individual. The camera is configured to acquire images of the floor marker, which has a known size or diameter, while the individual is standing. The processing unit is configured to capture an initial calibration image of the floor marker using the camera while an individual is standing still to determine the distance between the camera and the floor marker. The processing unit is further configured to capture subsequent time-varying images of the floor marker while the individual is standing (and swaying). Furthermore, the processing unit is configured to compare the calibration images to the subsequent time-varying images to determine a postural sway of the individual.

### CROSS-REFERENCE TO RELATED APPLICATIONS

The present application is related to and claims the priority benefit of U.S. Provisional Patent Application Ser. No. 62/593,679, filed Dec. 1, 2017, the contents of which is hereby incorporated by reference in its entirety into this disclosure.

### TECHNICAL FIELD

The present disclosure generally relates to a system and methods for collecting, calculating, and outputting data useful in analyzing an individual's postural sway.

### BACKGROUND

Medio-lateral (ML) and anterior-posterior (AP) sway balance assessment has been considered as a good indicator of the ability to body to stabilize its center of mass within the limits of the base of support. Impairments of the balance control, the result of a wide variety of neuromuscular and vestibular disorders, can lead to frequent falls and associated morbidity and mortality. The ML and AP sway assessment also provides valuable diagnostic and prognostic information on athletes suffering concussions. Current devices used to measure sway balance are mostly limited to laboratory settings and require trained personnel, hence, reducing their value for at-home or in-the-field assessment. For example, Optotrak Certus (NDI, Canada), widely considered as the gold-standard by the clinical and research communities, is expensive (tens of thousands of dollars), requires complex hardware, and is hard to operate without considerable training. Other alternatives in the market are those based on inertial sensors and pressure plates, but lack the accuracy and response speed for useful analysis. Therefore, improvements are needed in the field.

### SUMMARY

A postural sway analysis system is disclosed. The system includes a camera worn by an individual, a processing unit coupled to the camera, a floor marker placed on a floor near the shoes or feet of the individual. The camera is configured to acquire images of the floor marker, which has a known size or diameter, while the individual is standing. The processing unit is configured to capture an initial calibration image of the floor marker using the camera while an individual is standing still to determine the distance between the camera and the floor marker. The processing unit is further configured to capture subsequent time-varying images of the floor marker while the individual is standing (and swaying). Furthermore, the processing unit is configured to compare the calibration images to the subsequent time-varying images to determine a postural sway of the individual.

A method for acquiring postural sway of an individual is also disclosed. The method includes capturing a calibration image from a floor marker placed on a floor near the shoes or feet of an individual to determine the distance between the camera and the floor marker, wherein the calibration image is obtained from a camera worn by the individual. The method also includes capturing subsequent time-varying images from the floor marker while the individual is standing (and swaying). Furthermore, the method includes comparing the calibration image to the subsequent time-varying images to determine a postural sway of the individual.

### BRIEF DESCRIPTION OF THE DRAWINGS

In the following description and drawings, identical reference numerals have been used, where possible, to designate identical features that are common to the drawings.

FIG. 1A is a diagram showing a postural sway analysis system according to one embodiment.

FIG. 1B is a diagram showing a patient operating the postural sway analysis system of FIG. 1A.

FIG. 2 is a diagram illustrating anterior/posterior (AP) sway and medial/lateral (ML) postural sway displacement of a patient.

FIG. 3 is a diagram showing a method for processing postural sway information according to one embodiment.

FIG. 4A is a graph showing a comparison of ML sway data produced by a Vicon system and the system of FIG. 1A.

FIG. 4B is a graph showing a comparison of AP sway data produced by a Vicon system and the system of FIG. 1A.

FIG. 4C is a graph showing a comparison of both ML and AP sway data produced by a Vicon system and the system of FIG. 1A.

FIG. 5A is a graph showing a comparison of AP postural sway data for an Optotrak system and the system of FIG. 1A.

FIG. 5B is a graph showing a comparison of ML postural sway data for an Optotrak system and the system of FIG. 1A.

FIG. 6A is a graph showing a comparison of the AP mean absolute mean power frequency error for for an Optotrak system and the system of FIG. 1A.

FIG. 6B is a graph showing a comparison of the ML mean absolute mean power frequency error for for an Optotrak system and the system of FIG. 1A.

FIG. 7A is a graph comparing RMS AP displacement data of a Vicon system and the system of FIG. 1A.

FIG. 7B is a graph comparing RMS AP displacement data of a Vicon system and the system of FIG. 1A.

FIG. 8 is a table summarizing various abbreviations.

The attached drawings are for purposes of illustration and are not necessarily to scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In response to the need for a more efficient and effective postural sway analysis system, disclosed herein is a novel postural sway analyzer that can measure postural sway using an imaging system, processing unit, and a camera feature in a processing unit such as a smart cellular phone.

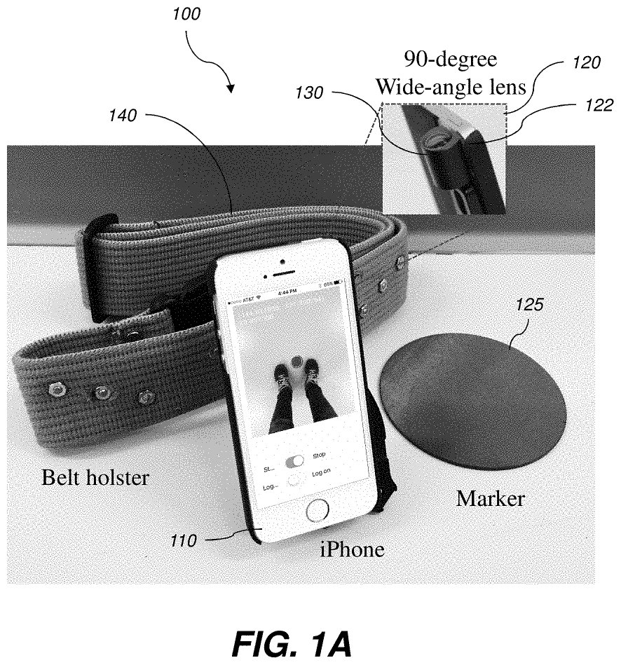

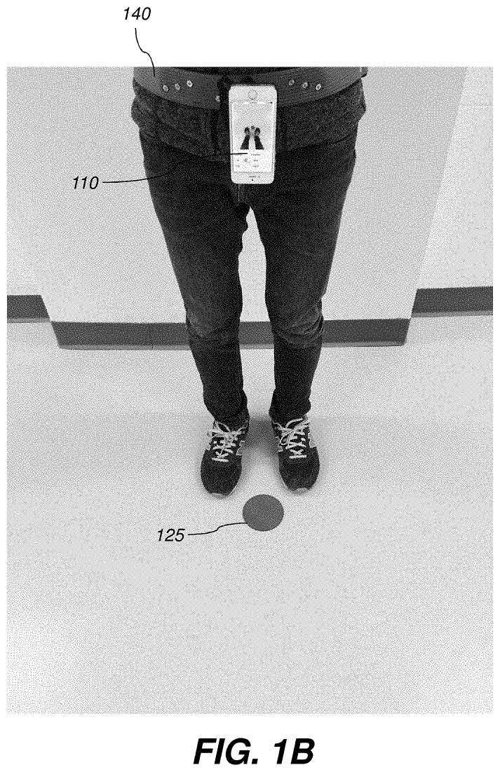

Referring to FIG. 1, a sway analysis system 100, according to the present disclosure is provided. The sway analysis system 100 generally includes a processing unit 110 and an imaging system 120. The system also includes a marker 125, which has a known size or diameter for calibrating the system 100 as described below. The processing unit 110 can be a general purpose processing unit, e.g., a smart cellular phone, such as an APPLE IPHONE, or other processing units, e.g., a special purpose processing unit such as an embedded system paired with an external mountable camera/lens systems. Various embodiments are within the scope of this disclosure. For example, a processing unit may be worn on a subject along with a camera as part of an imaging system capable of obtaining video where the processing unit can process real-time video and any post-processing of data or a separate external processing unit in communications (wireless or wired) with the on-subject processing unit for the purpose of post-processing of data, where the on-subject processing unit is coupled to the camera in a wired or wireless manner; or a wireless or wired camera as part of an imaging system can be worn on the subject while the processing unit(s) is off the subject but in electronic communication (wireless or wired) with the camera. In the latter embodiment, the camera may be configured to communicate image data directly to the processing unit, or indirectly by first recording the image data on a memory device to be used by the processing unit at a later time. Therefore, while the processing unit 110 is shown to be coupled to the imaging system 120, in certain embodiments these units may be only coupled electronically and not physically in contact with each other.

The processing unit 110 includes a processor (not shown) or multiple processors (not shown), memory (not shown), input/output (I/O) circuitry (not shown), and other peripheral circuits typically available in a smart cellular phone. The I/O circuitry may include a wireless communication circuit (not shown), e.g., a Bluetooth system or WiFi, and/or a wired communication circuit (not shown).

The imaging system 120 includes a camera 122 and a right angle lens assembly 130. It should be noted that the right angle lens assembly 130 may be avoided with the camera 122 placed in a manner in which it is pointed downward toward the shoes/feet of the subject. The camera 122 is typically integrated with the processing unit 110 but can also be part of the right angle lens assembly 130. The right angle lens assembly 130 includes a housing and a lens. The right angle lens assembly 130 is configured to transfer images from the lens to the camera 122 in a right angle manner. In the embodiment shown in FIG. 1 is where the right angle lens assembly 130 is fixedly coupled to the processing unit 110. The processing unit 110 may also be fitted with a belt strap 140 and/or a flexible arm holder for coupling the processing unit to a subject's belt.

The right angle lens assembly 130 is configured to tilt the view by 90 degrees and offer a wide angle of view. The camera 122 with the detachable right-angle lens is thus capable of capturing images of a subject's shoes/feet. Once worn, the camera angle can be adjusted, if needed, to bring the marker into a direct field of view and centering it on the camera screen

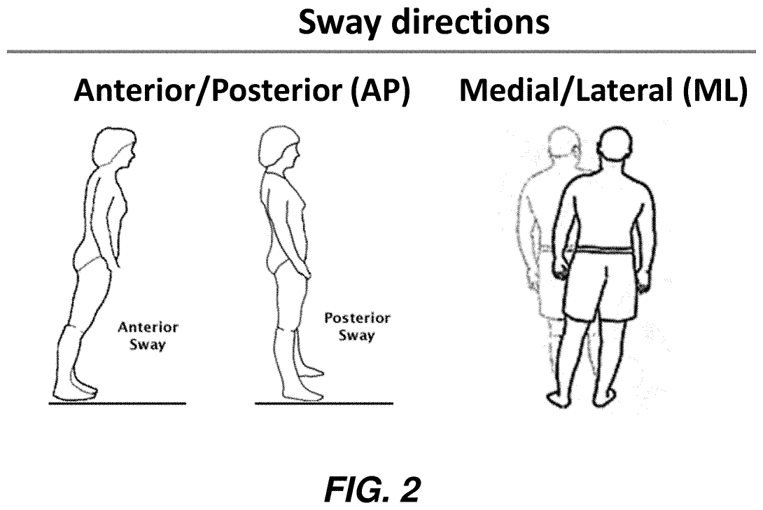

To analyze the postural sway of a subject, several parameters need to be monitored. Referring to FIG. 2, some of these parameters are depicted, including the anterior/posterior (AP) variation and the medial/lateral (ML) variation.

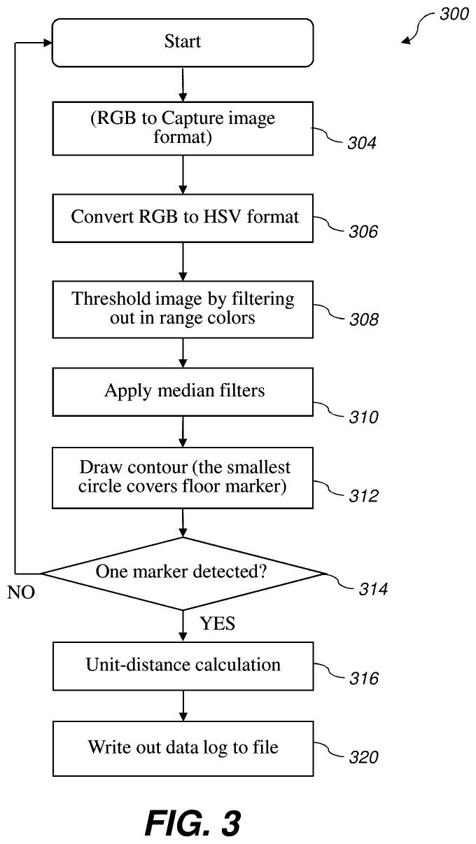

FIG. 3 shows a process 300 for analyzing postural sway using the system 100 according to one embodiment. The process is implemented by software running on the processing unit which identifies the floor marker from background and provides a time series data that quantifies sway motion. The process begins by a subject first activating the using a user interface of the processing unit (e.g., a touchscreen interface). The processing unit receives red-green-blue (RGB) image data from the camera (stage 304). The RGB data is then converted by the system to HSV (hue, saturation, and value) format for increased accuracy (306). The HSV image is them optionally processed to remove pixel data which is not within a predetermined color range (e.g., colors which are not in the range of the color of the marker) to further improve detection accuracy (stage 308). Next, the system filters the HSV image in a range of specific HSV values which is preset for the color of the marker (green in the illustrated embodiment, but other colors may be used) before converting it to a monochrome image that results in a white marker and black background. A median filter may then also be optionally applied to the image to reduce false positive marker recognition (stage 310). The processing unit then detects the boundary of the marker in the image and determines a center position of the marker in the pixel grid of the image (stage 312). If more than one marker is detected, the system assumes a false detection has occurred and moves to a successive image to attempt to recognize the marker again (decision block 314).

Once a successful marker image is detected, the system determines a calibration factor by performing a unit-distance calculation using the known size or diameter of the marker to determine a distance-per-pixel for the received image (stage 316). Successive images are then compared by the processing unit to determine the movement of the marker within the pixel grid of the received images. The movement of the floor marker within the image pixel grid is then used to determine the ML and AP sway of the user, since the marker movement is directly related to the movement of the camera relative to the marker. The marker movement data is then written to a data log file in the processing unit memory (stage 320) for further processing and output to a display (e.g., smartphone screen or other electronic display).

The sway data log records various parameters, including date, time, sampling frequency, floor marker size (unit distance), AP sway (distance) and ML sway (distance). In the software developed for the system of the present disclosure, sway assessment provides a brief on-screen summary for the users.

The sway data that is acquired from sway analysis system 100 of the present disclosure can be used to predict user health, as there is a known association between sway variables and various health conditions and diseases, such as concussion. The data acquired by the sway analysis system 100 can be stored and compared to a library of known parameters associated with such health conditions. The individual's values will be compared to these libraries to determine if any of the parameters exceed the threshold. If the threshold is exceeded on one or more parameters, the individual will be identified as being at higher risk for the associated health conditions.

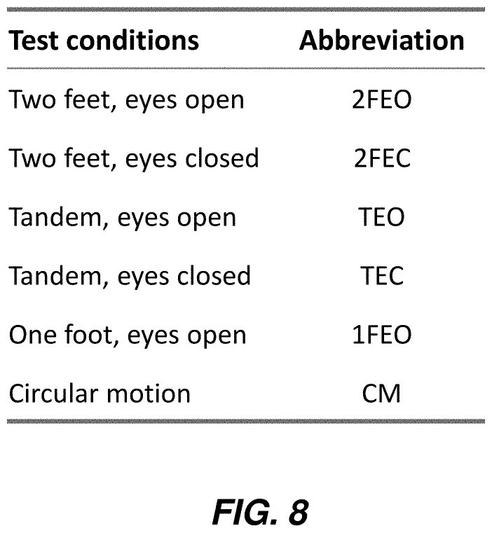

In one example, the system 100 was validated by its direct comparison to the optical motion analysis system, OptoTrak (Optotrak 3020, NDI), with an infrared LED of the OptoTrak placed on the wide-angle lens. Ten young healthy adults (24.6±3.4 yrs) were asked to stand quietly for 1 minute in the following conditions: on two feet with eyes open (2FEO), on two feet with eyes closed (2FEC), on one foot with eyes open (1FEO), and tandem standing eyes open (TEO). FIG. 8 summarizes these abbreviations. At the beginning of each trial, subjects were asked to perform an intentional big sway motion for accurate synchronization of the two systems. For data comparison from each system, spatial and temporal parameters were assessed and following statistical parameters were calculated: root mean square (RMS) and mean power frequency (MPF). Agreement between SwayWatch and Optotrak was evaluated with absolute error and Intra-Class Correlation (ICC (2,1)).

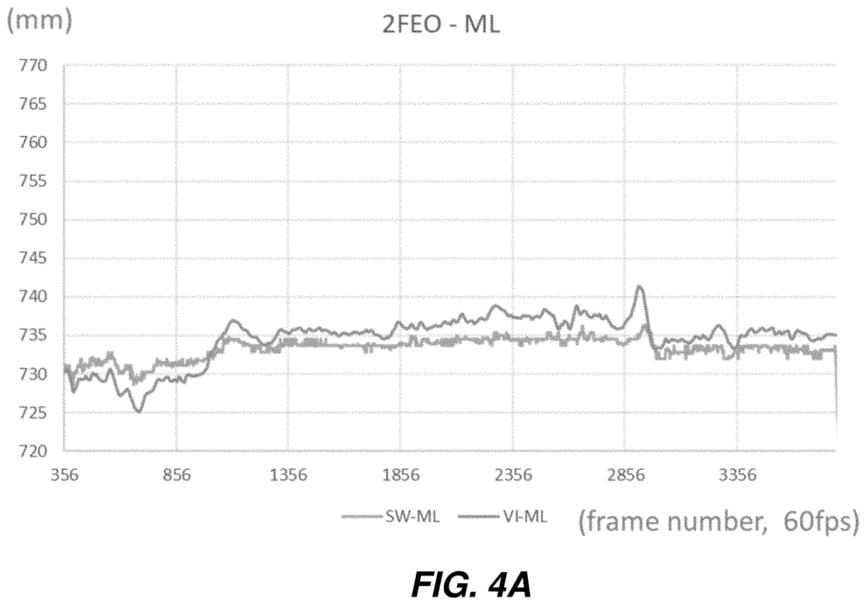

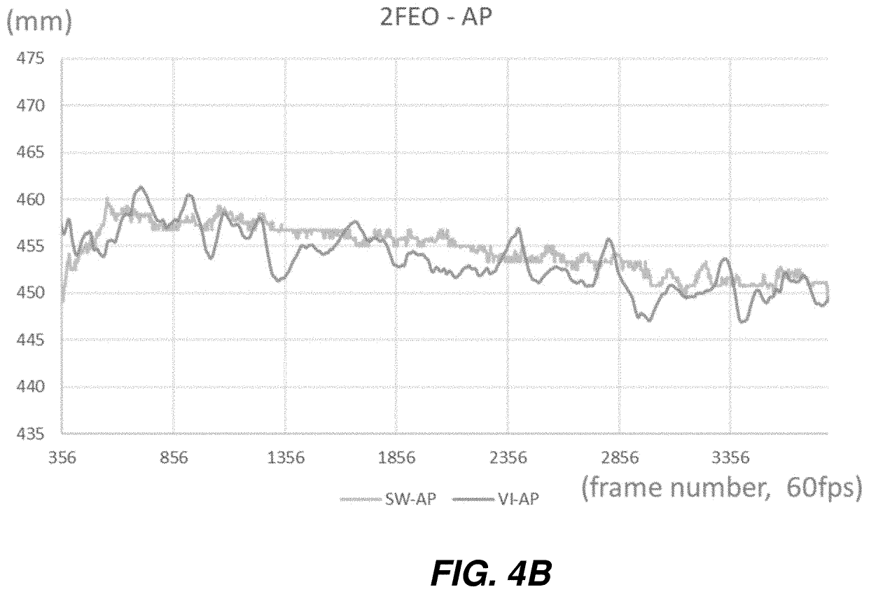

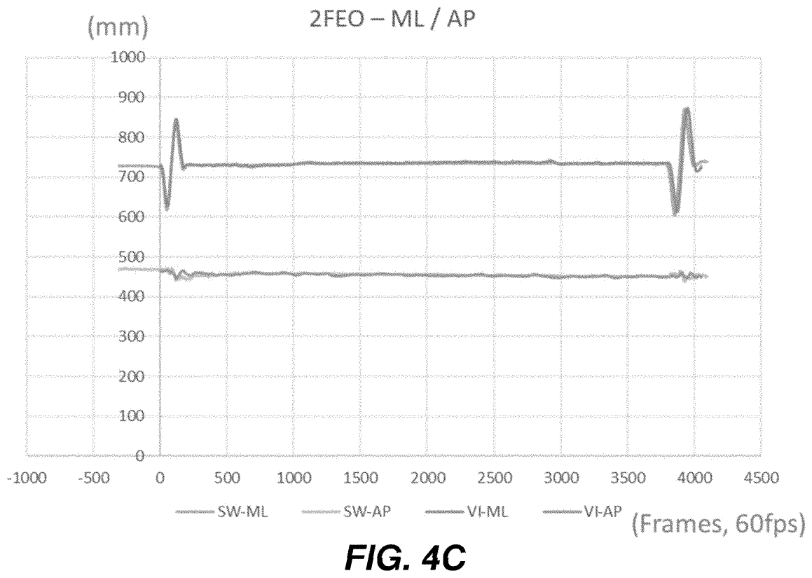

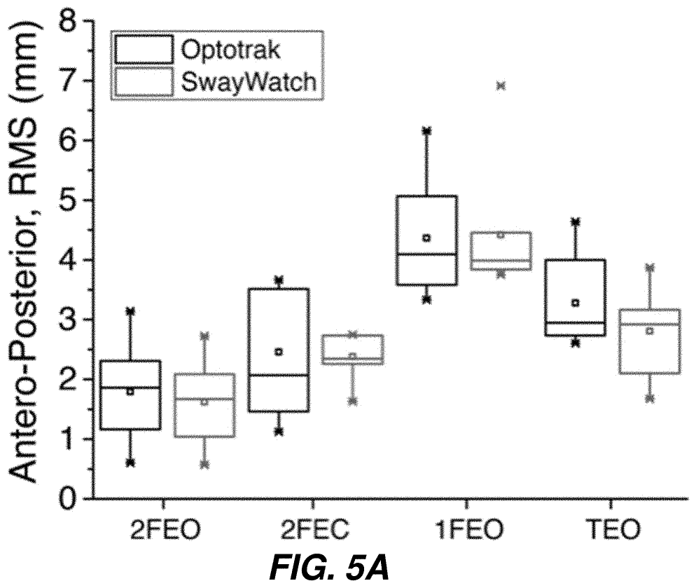

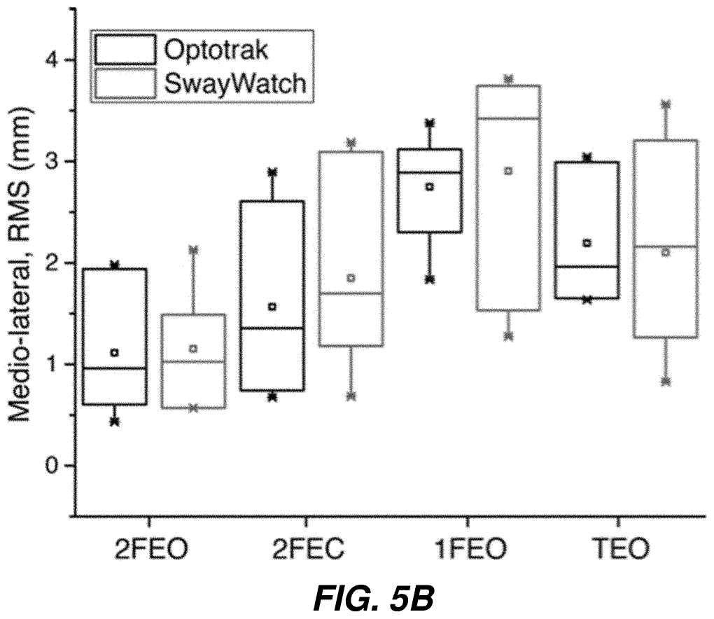

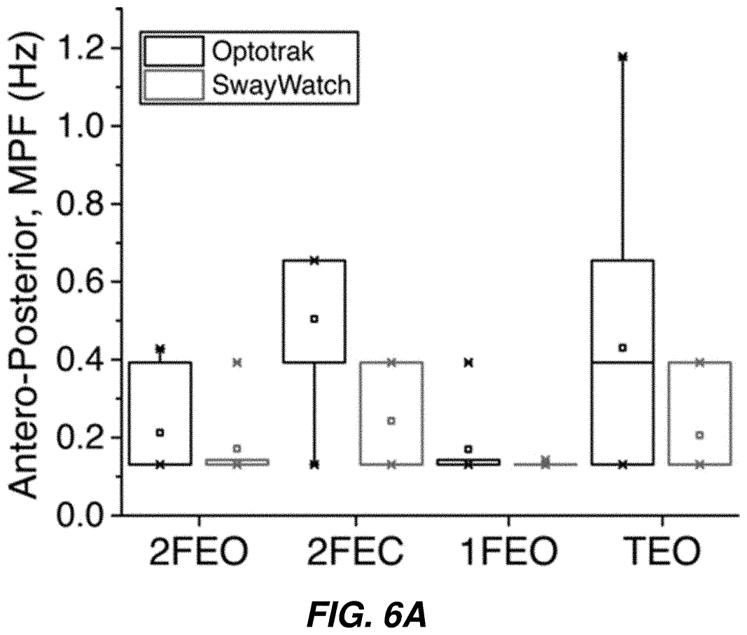

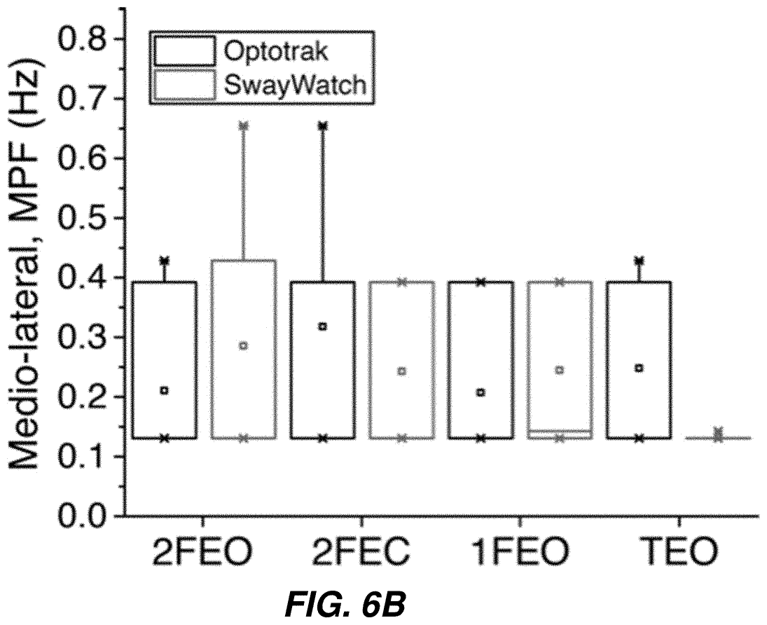

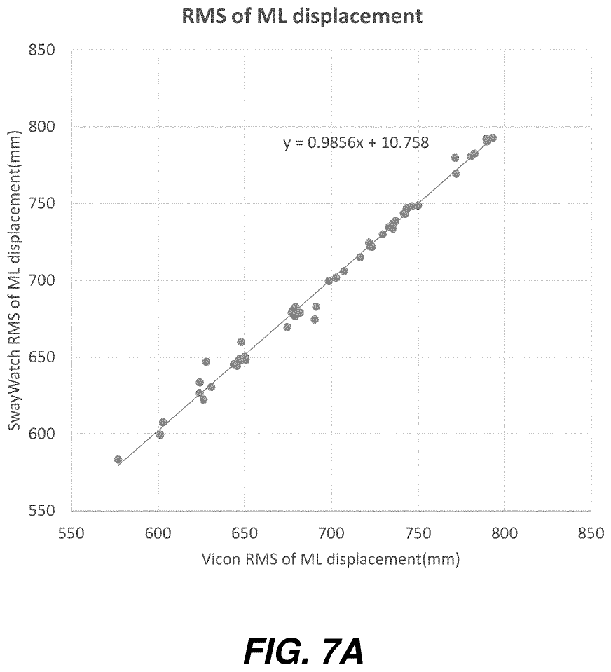

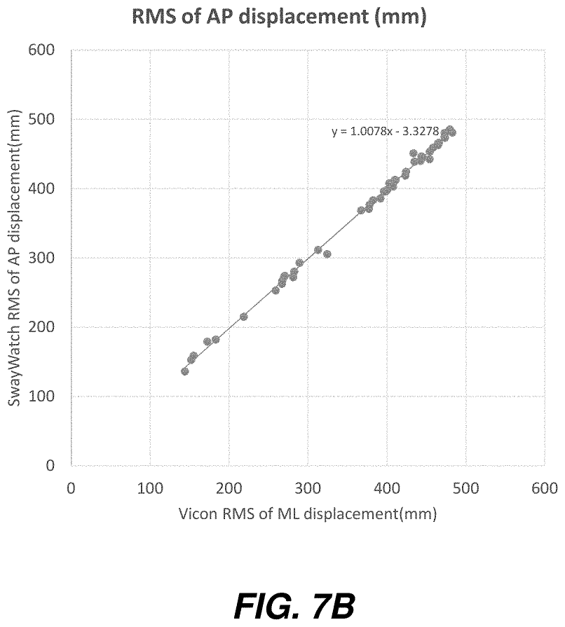

FIGS. 4A, 4B and 4C shows the displacement comparison of both a Vicon system and and the presently disclosed SwayWatch system 100. This comparison is analyzed in two different statistical model as following. In the illustrated example, the absolute RMS error was less than 1 mm in AP/ML: 0.3±0.3 mm/0.2±0.2 mm (2FEO), 0.5±0.4 mm/0.5±0.5 mm (2FEC), 0.6±0.3 mm/0.6±0.3 mm (1FEO), and 0.5±0.4 mm/0.5±0.4 mm (TEO). The ICCs for RMS as compared to an Optotrak system in AP/ML were: 0.92/0.93 (2FEO), 0.78/0.82 (2FEC), 0.86/0.75 (1FEO), and 0.74/0.86 (TEO), as shown in FIG. 5A and 5B. This result demonstrated an excellent level of agreements which means SwayWatch and OptoTrak (Optotrak 3020, NDI) systems show similar accuracy. The mean absolute MPF error was less than 0.4 Hz in AP/ML: 0.12±0.14 Hz/0.30±0.38 Hz (2FEO), 0.33±0.20 Hz/0.19±0.20 Hz (2FEC), 0.04±0.09 Hz/0.07±0.13 Hz (1FEO), and 0.30±0.28 Hz/0.08±0.13 Hz (TEO) for anterio-posterior/medio-lateral sways, respectively, as shown in FIGS. 6A and 6B. This also shows that each system shows moderate agreement from each other. And accordant ICCs in AP/ML planes were: 0.79/0.70, 0.62/0.68, 0.72/0.85, and 0.80/0.72, FIGS. 7A an 7B.

Those skilled in the art will recognize that numerous modifications can be made to the specific implementations described above. The implementations should not be limited to the particular limitations described. Other implementations may be possible. While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

1. A method for acquiring postural sway parameters of an individual, comprising:
capturing at least one calibration image from a floor marker placed on a floor near an individuals feet while an individual is standing, the calibration image obtained from a camera worn by the individual;
capturing subsequent time-varying images from the floor marker while the individual continues to stand; and
comparing the time time-varying images by a processing unit that is coupled to the camera to determine changes in position of the camera relative to the foot marker as a function of time to analyze postural sway of the individual.

2. The method of claim 1, wherein the camera is an integral part of the processing unit.

3. The method of claim 1, wherein the camera physically coupled to the processing unit.

4. The method of claim 1, wherein the camera electronically coupled to the processing unit.

5. The method of claim 4, wherein the coupling is by a wireless channel.

6. The method of claim 1, wherein the floor marker includes at least one identifiable feature such that the floor marker can be identified in the images.

7. The method of claim 6, the identifiable feature is a color.

8. The method of claim 7, the color is a shape.

9. The method of claim 6, wherein the identifiable feature is a color and shape combination.

10. A method for determining an individual's health condition, comprising:
gathering real-time postural sway parameter data from an individual;
comparing the postural sway parameter data to a library of known values; and
generating a postural sway variance to thereby identify an individual's risk of falling.

11. The method of claim 11, the postural sway parameter data comprises anterior/posterior variation and medial-lateral variation.

12. The method of claim 11, further comprising predicting an individual's risk of concussion using the postural sway parameter data.

13. The method of claim 11, the prediction of the individual's health condition is communicated to the individual using a processing unit.

14. The method of claim 14, the processing unit is a smart cellular phone.

15. A postural sway analysis system, comprising:
a camera worn by an individual;
a processing unit coupled to the camera;
a floor marker placed on a floor near the feet of the individual; and
wherein the camera is configured to acquire images from the floor marker as the individual is standing,
wherein the processing unit is configured to
capture at least one calibration image from the floor marker while an individual is standing,
capture subsequent time-varying images from the floor markers while the individual is standing, and
compare the calibration image to the subsequent time-varying images to determine movement of the camera relative to the floor marker as a function of time to analyze postural sway of the individual.

16. The postural sway analysis system of claim 16, the camera is an integral part of the processing unit.

17. The postural sway analysis system of claim 16, the camera physically coupled to the processing unit.

18. The postural sway analysis system of claim 16, the camera electronically coupled to the processing unit.

19. The postural sway analysis system of claim 19, the coupling is by a wireless channel.
